(19) United States
(12) Patent Application Publication
(10) Pub. No.: US 2007/0134792 A1
(43) Pub. Date: Jun. 14, 2007

(54) Stem Cells and Signals Developed for Use in Tissue and Organ Repair and Replacement

(75) Inventors: Jianwu Dai, Boston, MA (US); Eugene Bell, Boston, MA (US)

Correspondence Address: FISH & RICHARDSON PC, P.O. BOX 1022, MINNEAPOLIS, MN, 55440-1022, US

(73) Assignee: TEI Biosciences, Inc.

(21) Appl. No.: 11/678,143

(22) Filed: Feb. 23, 2007

Related U.S. Application Data
(63) Continuation of application No. 10/400,753, filed on Mar. 27, 2003.
Application No. 10/400,753 is a continuation of application No. 10/005,053, filed on Dec. 4, 2001, now abandoned.
(60) Provisional application No. 60/251,125, filed on Dec. 4, 2000.

(51) Int. Cl. C12N 5/08; C12N 5/06

(52) U.S. Cl. 435/325; 435/366

(57) ABSTRACT

Methods and compositions for repairing tissue. Certain embodiments of the invention involve transdifferentiation of cells in a manner not heretofore provided for. One embodiment of the invention features methods for producing stem cells. These methods can involve exposing cells (e.g., human fibroblasts) to a processed or activated egg extract (e.g., activated egg extract); and culturing the cells for a period of time to become stem cells. A cell culture can be performed in two or three dimensions, so that organ tissue or whole organs may be produced, e.g., for transplantation. Another embodiment of the invention features methods for promoting wound healing by using signaling complexes.

### RELATED APPLICATIONS

This application claims priority from provisional application No. 60/251,125, filed Dec. 4, 2000, the entire contents of which is incorporated herein by reference. This application is related to copending U.S. application Ser. Nos. 09/672,686, filed Sep. 28, 2000, the entire contents of which is incorporated herein by reference.

### BACKGROUND OF THE INVENTION

The need to replace tissue that has been lost to disease, injury, or as a result of surgical intervention has been a long-standing one. Needed for the task of rebuilding tissues for implantation are cells, signals and scaffolds which when combined provide a tissue or organ primordium which lends itself to vascularization, remodeling and reconstitution of a functional replacement for the body part required. Examples of tissues and organs that can be built as prosthetic devices for transplantation include nervous tissue, skin, lens, vascular tissue, cardiac tissue, pericardial membrane, bone cartilage, tendon, ligament, and organs such as kidney, liver, glands, urological tissues and intestinal tissues. Ideally the cells needed for the reconstitution of a replacement part for the body are pluri- or multipotent, able under it influence of appropriate signals to become, predictably, the tissue required to restore lost function. A variety of scaffolds have been used in tissue engineering, the most promising of which are based on the use of the family of collagen molecules, formed into fibers, in imitation of their structure in actual tissues.

The availability of stem cells for use in tissue engineering is stringently limited since cloning of the human egg has gained only minimal acceptance because of perceived ethical considerations. Matching the genotype of an individual in need of a prosthetic device would require the use of enucleated eggs supplied with nuclei from cells of the potential graft recipient. The procedure is costly because of the need to use donated eggs from an appropriate female entailing certain health risks. Another approach consists of harvesting egg cytoplasm, responsible for reprogramming a post-natal cell nucleus, preferably from a mammal, although egg cytoplasm from lower vertebrates is also possible as described by Wangh in U.S. Pat. No. 5,651,992. A reprogramming extract can have the same effect on a nucleus from an individual needing a graft, as the cytoplasm of an intact egg from which the nucleus is removed.

### BRIEF DESCRIPTION OF THE INVENTION

Stem cells are generated by culturing any type of cell that can be removed from a donor, in the presence of an animal egg extract or fraction thereof. In a preferred embodiment, the cells are human fibroblasts. Methods for identifying signaling complexes that can direct differentiation of stem cells and/or transdifferentiation of cells that are not stem-like cells into specific cell types, tissues, and organs are described herein.

### DETAILED DESCRIPTION OF THE INVENTION

I. Generating Stem Cells by Dedifferentiating Pre- or Post-Natal Cells with Processed or Activated Egg Extracts

The term “extract” includes any composition or mixture derived from breaking, lysing, or homogenizing a cell. An extract may be subjected to fractionation as described herein. Fractionated extracts may also be referred to herein as “extracts”. Preferably, an extract of the invention contains no cellular membranes or nucleic acids (e.g., DNA or RNA). In certain embodiments, extracts may include signaling complexes described herein.

In one embodiment, the extract is derived from processed or activated egg cells from a vertebrate animal, preferably from a mammal (e.g., a cow or pig). Extracts and extract fractions of the eggs may be prepared by methods known in the art. Similarly, methods known in the art for the activation of eggs may be used (Gerhart et al. (1984) J. Cell Biol. 98:1247). For example, egg activation can be achieved by application of two 1.0 kV/cm DC electric pulses for 60 μseconds each at a 5 second interval in an activation medium containing 0.3 M d-sorbitol, 0.1 mM MgSO₄, and 0.05 M CaCl₂ (Polejaeva et al (2000) Nature 407:85).

Processed or activated eggs are then suspended in a buffer solution, including, but not limited to Tris buffer, HEPES buffer or preferably phosphate buffered saline (PBS) over a pH range of 4.0-11.0 but preferably at 7.4. Preferably, the buffer is kept at a temperature of about 4° C. The buffer may include protease inhibitors. Cellular membranes are disrupted, for example, by mechanical forces such as those produced by ultrasound treatment. The sample containing activated egg cytoplasm in a buffer solution is subjected to centrifugation (for example, at 17,000 g for 20 minutes) to remove plasma membranes and particles, particularly the nuclei and mitochondria. After centrifugation, pelleted solid particulate matter is discarded, while the liquid supernatant is retained as the extract.

Preferably, the extract contains no mitochondria or mitochondrial DNA. Mitochondrial contamination in the final extract can be detected, for example, by staining the extract with a mitochondrial specific dye such as JC1 or by carrying out a polymerase chain reaction (PCR) using mitochondrial DNA specific oligonucleotide primers to determine whether mitochondrial DNA is present. PCR may also be used to determine if there is residual mitochondria DNA contamination. The active egg extract is the centrifugation supernatant free of DNA.

A processed or activated egg extract may be subjected to one or more further fractionation techniques like chromatographic or separation techniques known in the art such as ion exchange (e.g., anion or cation exchange) chromatography, gel filtration chromatography, affinity chromatography, high-performance liquid chromatography (HPLC), capillary electrochromatography (CEC), gradient (e.g., glycerol or sucrose gradient) centrifugation, two-dimensional gel electrophoresis, immunoprecipitation, dialysis, and ammonium sulfate precipitation.

In practice, the stem cells of the invention are generated by culturing any type of cell that can be removed from a donor, in the presence of an animal egg extract or fraction thereof. In a preferred embodiment, the cells are human fibroblasts. The cell may be exposed to an animal egg extract using any of a number of methods. In one embodiment, the extracts or fractions thereof are added directly to the culture medium in which the cell is maintained. In a preferred embodiment, the concentration of total protein in the culture medium is about 1-10 μg/ml. In a further embodiment, glass beads mixed with the cells may be used to facilitate entry of extract proteins into the cell (glass beads increase cell permeability by limited disrupt of the cellular membrane).

Extracts or fractions thereof of egg cytoplasm are added to the culture medium as described above, and the cell is cultured in a plate. Glass beads are then added to the culture plate. The glass beads are sterile, and are about 1 mm in diameter. The plate is then subjected to shaking. In a preferred embodiment, the plate is shaken for about 10-20 seconds. The shaking allows the glass beads to create breaks in the plasma membrane of the cell and allows the egg extract proteins to enter the cell directly. In another embodiment, an egg extract or fraction thereof may be microinjected directly into a cell. In still another embodiment, a detergent that can facilitate protein entry into the cell may be added to the culture medium.

After the cell is exposed to a processed or activated egg extract or fraction thereof, the cell is maintained in a defined culture medium (i.e., is cultured) for a period of time (preferably between about 10 days and 60 days). Upon completion of the culture period, the cell is assayed for a phenotype diagnostic for stem cells. In one embodiment, a cell may be assayed for the presence of the stem-cell-specific cell surface marker. In a preferred embodiment, the stem-cell surface marker is CD 34. In another embodiment, a cell may be assayed for the ability to differentiate (using any of the methods described herein) into a particular cell type. The term “dedifferentiate” refers to the process by which cell commitment to specific fates is reduced. Cells that are determined to be stem cells (e.g., those which express a stem-cell-specific cell surface marker such as CD 34 can be subcloned and expanded to provide a pool of stem cells.

II. Signaling-Complexes Designed to Induce Expression of Specific Phenotypes in Stem Cells.

Another embodiment of the present invention includes methods of generating and fractionating extract from donor animal tissues of porcine or bovine origin to promote cell division, morphogenesis, and differentiation of specific tissues and organs. During early development, animal tissues and/or organs contain specific pools of growth factors and other signaling molecules, referred to herein as “signaling complexes,” that can promote differentiation of specific cell, tissue and/or organ types. Signaling complexes are composed of one or more proteins that can specifically induce stem cells to express predictable phenotypes and are also able to induce transdifferentiation.

The source of the tissue used in producing the signaling complex may include, but is not limited to, pre- or post-natal mammals (e.g., pigs and cows). Any type of tissue, including but not limited to, nerve, brain, liver, muscle, heart, lung, cartilage, bone, tendon, pancreas, kidney or skin can be used. Preferred, non-limiting examples of procedures for preparing extract are as follows.

In one embodiment, tissue is extracted using buffer extraction. A specific tissue or organ is collected from pre- or post-natal animals, washed with buffer, and cut into small pieces. The buffer may be, for example, Tris buffer, HEPES buffer, or PBS, at a pH or 4.0-11.0, preferably 7.4., preferably includes EDTA (at for example, 0-10 mM, 0.5-5 mM, or preferably 2 mM), and may include protease inhibitors (for example, 1 mM PMSF and or 1 μM E-64). Preferably, the buffer is kept at about 4° C. The cut pieces of tissue are homogenized in buffer, preferably the same buffer used for washing, and extracts are obtained by collecting the supernatant after centrifugation.

Tissue may also be subjected to enzyme extraction. Enzymes are used to degrade the extracellular matrix (e.g., collagen) to release any signaling molecules that bind to the matrix. Homogenized tissue (e.g., skin) is incubated with an enzyme and then centrifuged to remove particulate matter. The extract is the supernatant obtained after centrifugation. A preferred, non-limiting example of enzyme extraction is as follows. Homogenized tissue (e.g., skin) is incubated with 180 U/ml hyaluronidase at room temperature for 1.5 hours, and then is incubated with 160 U/ml collagenase 4/3 for an additional 1.5 hours at room temperature.

Tissue may also be extracted by acid extraction to recover signaling molecules that are soluble at low pH. A preferred, non-limiting example is as follows. 0.2 ml of 1 N HCl is added to each ml of the homogenized tissue (e.g., skin), which is then stirred for 30 minutes at room temperature. The extract is neutralized with NaOH. Other acids may also be used.

The extracts may be used directly or can be subjected to one or more further fractionation techniques, for example any of the chromatographic or separation techniques known in the art, including ion exchange (e.g., anion or cation exchange) chromatography, gel filtration chromatography, affinity chromatography, high-performance liquid chromatography (HPLC), capillary electrochromatography (CEC), gradient (e.g., glycerol or sucrose gradient) centrifugation, dialysis, two-dimensional gel electrophoresis, immunoprecipitation, and ammonium sulfate precipitation. Both extracts and fractions can be stored or used, for example in the form of a solution or a lyophilized powder. Extracts of fetal tissues, e.g. so prepared have been shown to induce, predictably, desired phenotypes in stem cells.

In one embodiment, pluripotent murine embryonic stem (ES) cells are used to assay tissue extracts and/or fractions thereof for the ability to direct the differentiation of ES cells into specific cell types. ES cells are first predifferentiated into embryoid bodies (EBs) using methods well known in the art. The cells of the undifferentiated EBs are then dissociated and cultured in the presence of various tissue extracts and/or fractions thereof. EB cells may be cultured as a monolayer culture or in suspension. In a preferred embodiment, the EB cells are cultured in three dimensions, for example in a collagen scaffold in, e.g., defined medium or low serum medium. The period of time the EB cells are cultured may range from about 1 week to about one month, or longer than one month.

At the end of the culture period, the cells are assayed for specific cell types including, but not limited to, heart, muscle cells, nerve cells, insulin-secreting cells, hepatocytes, kidney, lung, cartilage and bone cells. In one embodiment, the cells may be assayed for the presence of one or more tissue specific cell surface markers, for example, by immunofluorescence. In another embodiment, the cells may be assayed for expression of one or more tissue specific mRNAs using methods well known in the art, Northern blotting or RT-PCR.

Tissue extracts or fractions thereof which can induce differentiation of EB cells into specific cell types are identified as signaling complexes which can be used in the methods of the invention to induce differentiation of stem cells into specific cell types, tissue, and/or organs, as well as to induce transdifferentiation of non-stem cells. In a further embodiment, stem cells produced by the methods described herein may be used interchangeably with EB cells. When using stem cells to identify signaling complexes, the stem cells do not need to be predifferentiated into EBs.

III. Use of Signaling Complexes to Induce Transdifferentiation.

Another embodiment of the present invention is the use of animal tissue extracts and fractions for cell transdifferentiation. “Transdifferentiation” includes a change of a cell or tissue from one differentiated state to another. Signaling complexes and/or fractions thereof can be used to direct stem cells and/or differentiated adult cells into different cell types, tissues, and/or organs using animal tissue extracts and/or fractions thereof.

In an exemplary, non-limiting embodiment, human fetal skin fibroblasts can be transdifferentiated into heart, muscle, nerve, liver, kidney, insulin-secreting, lung, cartilage and bone cells with the above described signaling complexes. Fibroblasts are first isolated from 8-24-week human fetal skin from medically approved aborted fetuses. After 2 passages, the cells are then cultured in three-dimensional collagen scaffold with either low serum medium or defined medium with the addition of a signaling complex of a total protein concentration of 10 μg/ml to 50 μg/ml. The culture medium is changed every 3-4 days. After about a week to about one month in culture, morphological changes can be observed in the cells; RT-PCR and/or immunostaining can be used to characterize expression of numerous phenotypes, each induced by a specific signaling complex including heart, cartilage, bone, endocrine pancreas, liver, and lung, for example. Specifically, muscle actin is one of the markers for cardiogenic cells, and insulin is a marker for insulin-secreting cells.

Adult stem cells (e.g., those produced by the methods of the invention) and in addition, umbilical cord, bone marrow cells, adipocytes, and many differentiated cells (not limited to fibroblasts) can be induced to differentiate predictably using the methods described herein. The cells may be cultured using various culture methods, for example, monolayer culture, suspension culture, and three-dimensional culture. Signaling complexes can be applied in vitro (e.g., added to culture), and may also by applied in vivo (e.g., added during transplantation of tissue) or as pharmaceutical agents.

IV. Use of Signaling Complexes for Wound Healing and Tissue Repair.

In another embodiment, signaling complexes and/or fractions thereof can be used for wound healing and tissue repair. As used herein, the term “wound” includes any cut, abrasion, burn, puncture, tear, break, fracture, or other tissue injury, loss of tissue integrity, or diminution of function. For example, skin tissue extracted with Tris-buffer (pH 8) yields an extract that can be used to treat topical wounds (e.g., skin wounds). Extracts and/or fractions thereof are delivered to the wound in a carrier, for example, a cross-linked collagen scaffold, a collagen foam, or injectable collagen fiber (see U.S. Pat. Nos. 5,800,537; 5,709,934; 5,893,888; and 6,051,750; all of the (contents of which are incorporated herein by reference).

The carrier is hydrated with a liquid solution of the extract. Preferably, the total protein concentration ranges from 1.0 pg/ml to 10 mg/ml. In one embodiment, the treatment includes application of one or more grafts of the carrier containing the extract to treat a single wound. In another embodiment, one graft is used, and multiple doses of the extract can be given by successive applications or injections to the graft. In the practice of the invention, one application of signaling complex results in highly significant reduction of wound contraction in a rat model, compared with control grafts that have not received signaling complexes.

Single extracts, fractions thereof, and/or any combinations thereof may be used for one kind or several kinds of wound healing or tissue replacement. Extracts of signaling molecules and/or fractions thereof made using the methods of the instant invention can be used to treat numerous types of wounds, to promote, for example, bone regeneration or tendon repair and is not limited to topical wounds.

V. Use of ECM Particulates as Sources of Signaling Complexes

In another embodiment, the present invention provides a method for tissue and organ regeneration using extracellular matrix (ECM) particulates (see U.S. Pat. Nos. 5,893,888; 5,800,537; and 6,051,750, and U.S. patent Ser. No. 09/511,433, filed Jun. 23, 2000, all of the contents of which are herein incorporated by reference), derived from tissues noted above but not limited to them, and extracts and/or fractions of the foregoing to induce expression of specific tissues or organs. The method consists of two major steps: 1) generation of primordia with tissue specific stem cells or transdifferentiated cells in vitro incorporated into two or three-dimensional scaffolds with signaling complexes, and 2) transplantation of the primordia into animals (e.g., humans) for in vivo tissue development and regeneration. The method includes the repair and/or regeneration of many types of tissues and organs (e.g., skin, liver, kidney, pancreas, blood vessel, bone, cartilage, ligament, and tendon).

When the cells are properly differentiated into tissue specific cells, vascularization is critical to the success of the tissue. In one embodiment, a specific signaling complex that promotes capillary formation in vitro is used. In another embodiment, a scaffold is implanted into an animal host or directly into a human, at an early stage of development, in the form of a primordium, to allow for vascularization and subsequent growth and maturation under native conditions.

In vitro differentiation is carried out by culturing stem cells or induced stem cells in three-dimensional collagen scaffolds with the addition of specific signaling complexes. The scaffolds can be cross-linked and freeze-dried collagen or collagen fiber, collagen gel, a collagen-gel mixture, or any of these with the addition of different types of collagen, or the addition of other types of proteins or polymers such as gelatin. The collagen scaffolds can be cross-linked or non cross-linked. The cross-linking procedure is done by using a variety of chemical cross-linkers or by physical approaches such as UV irradiation. Thus, different types of scaffolds with different mechanical properties can be prepared for different types of tissue regeneration.

The scaffolds not only provide a three-dimensional structure for the cells to attach to and grow, but, being fibrous, they resemble the native environments for cells sense as they differentiate and undergo tissue development under the influence of the tissue specific signaling complexes. Cells may be added to freeze-dried scaffolds by hydrating them with a cell suspension (e.g., at a concentration of 100 cells/ml to several million cells/ml). Incorporation of cells into other types of scaffolds is done by adding cells to a collagen solution, preferably at 4° C. The methods of adding the signaling complexes vary. The extracellular matrix microparticulates can be added, for example, when the freeze-dried scaffolds are manufactured or tissue extracts or fractions thereof are added to the culture or scaffold directly.

Low serum medium or defined medium is used for in vitro stem cell differentiation or cell transdifferentiation. The culture time may vary from about 10 days to about 60 days. Cells are characterized by morphology by ELISA, by RT-PCR and/or by immunostaining to screen for cell-type-specific markers. For tissue regeneration using small scaffolds (<100 cubic millimeters in size), the medium is changed manually, and the signaling complexes are added every 3-4 days. For larger scaffolds, the culture is maintained, for example, in a bioreactor system. The system is designed to use a minipump for medium change. The pump is operated in the incubator. Scaffolds are kept in a special container with two tubes connected to the pump. Out of the scaffold container, fresh medium is mixed with the medium pumped out. The medium pumped back to the container will container about 5% fresh medium. This ratio varies from about 1% fresh medium to about 50% fresh medium. When signaling complexes are added, 100% fresh medium containing these signaling complexes will be added to the scaffold. The pump rate is adjusted to 0.1 ml/min or slower. The medium delivery system can be tailored to the type of tissue being manufactured. All culturing is performed under sterile conditions.

Equivalents

Those skilled in the art will recognize, or be able to ascertain using no more than routine experimentation, numerous equivalents to the specific procedures described herein. Such equivalents are considered to be within the scope of the present invention and are covered by the following claims. The contents of all references, issued patents, and published patent applications cited throughout this application are hereby incorporated by reference. The appropriate components, processes, and methods of those patents, applications and other documents may be selected for the present invention and embodiments thereof.

What is claimed is:

1. A method for producing stem cells comprising:
a) exposing cells to a processed or activated egg extract; and
b) culturing said cells for a period of time such that said cells dedifferentiate to become stem cells.

2. The method of claim 1, wherein said processed or activated egg extract is bovine, porcine or lower vertebrate derived.

3. The method of claim 1, wherein said period of time is between about 10 days and about 60 days.

4. The method of claim 1, wherein said exposing step comprises adding said processed or activated egg extract to a culture medium containing said cells.

5. The method of claim 4, further comprising the addition of glass beads to said culture medium.

6. The method of claim 4, further comprising the addition of a detergent to said culture medium.

7. The method of claim 4, wherein said culturing is performed in two dimensions.

8. The method of claim 4, wherein said culturing is performed in three dimensions by incorporating said cells into a scaffold.

9. The method of claim 8, wherein said scaffold is a cross-linked collagen scaffold, a collagen foam, or an injectable collagen fiber.

10. The method of claim 1, further comprising injecting said processed or activated egg extract into said cells.

11. The method of claim 1, wherein said cells are fibroblasts.

12. The method of claim 11, wherein said fibroblasts are human fibroblasts.

13. Stem cells produced by the method of claim 1.

14. A method for identifying a signaling complex comprising:
a) exposing an embryoid body cell or a stem cell to a signaling complex;
b) culturing said embryoid body cell or said stem cell; and
c) determining the effect of said signaling complex on the differentiation of said embryoid body cell or said stem cell into a desired cell type.

15. The method of claim 14, wherein said signaling complex is derived from pre- or post-natal tissue.

16. The method of claim 15, wherein said signaling complex is derived from nerve tissue, brain, liver, muscle, heart, lung, cartilage, bone, tendon, pancreas, kidney or skin.

17. The method of claim 14, wherein said culturing is performed in a collagen scaffold.

18. The method of claim 14, wherein said culturing is performed for a period of time between about 10 days and about 60 days.

19. A signaling complex identified by the method of claim 14.

20. A method for transdifferentiating cells into desired cell types comprising:
a) exposing cells to at least one signaling complex;
b) culturing said cells wherein said cells become the desired cell type.

21. The method of claim 20, wherein said cells are pre- or post-natal cells.

22. The method of claim 20, wherein said signaling complex is derived from nerve tissue, brain, liver, muscle, heart, lung, cartilage, bone, tendon, pancreas, kidney or skin.

23. The method of claim 20, wherein said signaling complex is produced by buffer extraction, enzyme extraction, or acid extraction.

24. The method of claim 20, wherein said signaling complex is combined with a second signaling complex derived from a different tissue.

25. The method of claim 24, wherein said signaling complex is incorporated into a scaffold selected from the group consisting of a cross-linked collagen scaffold, a collagen foam, and an injectable collagen fiber.

26. Cells transdifferentiated into desired cell types by the method of claim 20.

27. A method for promoting wound healing comprising exposing a wound to a signaling complex.

28. The method of claim 27, wherein said wound is a topical or internal wound.

29. The method of claim 27, wherein said signaling complex is derived from pre-natal, or post-natal tissue.

30. The method of claim 29, wherein said signaling complex is derived from nerve tissue, brain, liver, muscle, heart, lung, cartilage, bone, tendon, pancreas, kidney or skin.
